(19) United States
(12) Patent Application Publication
(10) Pub. No.: US 2024/0156518 A1
(43) Pub. Date: May 16, 2024

(54) MEDICAL SYSTEMS FOR ABLATION OR ELECTROPORATION INCLUDING A REMOVABLE ELECTRICALLY CONDUCTIVE STYLET AND METHODS OF USE

(71) Applicant: INTUITIVE SURGICAL OPERATIONS, INC., Sunnyvale, CA (US)

(72) Inventors: Serena H. Wong, Los Altos, CA (US); Samuel Raybin, San Jose, CA (US); Randall L. Schlesinger, San Mateo, CA (US)

(21) Appl. No.: 18/510,019

(22) Filed: Nov. 15, 2023

Related U.S. Application Data
(60) Provisional application No. 63/425,879, filed on Nov. 16, 2022.

(51) Int. Cl. A61B 18/14

(52) U.S. Cl. CPC A61B 18/1477; A61B 18/1492; A61B 2018/00184

(57) ABSTRACT

A medical system may comprise a flexible elongated tool in which a lumen extends. The lumen may be defined by an inner wall of the elongated tool. The medical system may also comprise a flexible stylet configured to extend within the lumen and contact the inner wall of the elongated tool at a plurality of points. Energy conducted through the stylet may be transmitted at the plurality of points to the elongated tool.

### RELATED APPLICATIONS

This patent claims priority to and benefit of U.S. Provisional Application No. 63/425,879, filed Nov. 16, 2022 and entitled “MEDICAL SYSTEMS FOR ABLATION OR ELECTROPORATION INCLUDING A REMOVABLE ELECTRICALLY CONDUCTIVE STYLET AND METHODS OF USE,” which is incorporated by reference herein in its entirety. This patent application is also related to U.S. Provisional Patent Application 63/425,973, entitled “MEDICAL SYSTEMS FOR ABLATION OR ELECTROPORATION INCLUDING AN EXPANDABLE ELECTRICALLY CONDUCTIVE STYLET AND METHODS OF USE,” filed Nov. 16, 2022, which is incorporated by reference herein in its entirety.

### FIELD

Examples described herein relate to medical systems for energized treatment, such as ablation or electroporation, including an elongated tool in which a removable electrically conductive stylet may be inserted. The stylet may be shaped to establish a plurality of contact points between the stylet and the elongated tool.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools may include instruments such as biopsy, ablation, electroporation, or other energy delivery instruments. Improved systems and methods are needed to allow minimally invasive tools to be used for multiple purposes such as biopsy procedures and energy delivery procedures.

### SUMMARY

The following presents a simplified summary of various examples described herein and is not intended to identify key or critical elements or to delineate the scope of the claims.

Consistent with some examples, a medical system may comprise a flexible elongated tool in which a lumen extends. The lumen may be defined by an inner wall of the elongated tool. The medical system may also comprise a flexible stylet configured to extend within the lumen and contact the inner wall of the elongated tool at a plurality of points. Energy conducted through the stylet may be transmitted at the plurality of points to the elongated tool.

Consistent with other examples, a method may comprise extending an elongated tool into a patient anatomy, extending a stylet within a lumen of the elongated tool, establishing a plurality of contact points between the stylet and an inner wall of the elongated tool and applying an electrical current to the stylet to conduct electricity from the stylet to the elongated tool.

Consistent with other examples, a method may comprise extending an elongated tool into a patient anatomy, extending a stylet within the elongated tool, delivering a conductive fluid into a lumen of the stylet and applying an electrical current to the stylet to conduct electricity through the conductive fluid to the elongated tool.

Other examples include corresponding computer systems, apparatus, and computer programs recorded on one or more computer storage devices, each configured to perform the actions of any one or more methods described below.

It is to be understood that both the foregoing general description and the following detailed description are illustrative and explanatory in nature and are intended to provide an understanding of the various examples described herein without limiting the scope of the various examples described herein. In that regard, additional aspects, features, and advantages of the various examples described herein will be apparent to one skilled in the art from the following detailed description.

### BRIEF DESCRIPTIONS OF THE DRAWINGS

FIG. 1 illustrates a medical instrument system including a delivery device and an elongated tool in which extends an electrically conductive stylet, according to some examples.

FIG. 2A is a cross-sectional side view of an elongated tool in which extends an electrically conductive stylet including a helical portion, according to some examples.

FIG. 2B is a cross-sectional side view of an elongated tool threadedly connected with an electrically conductive stylet including a helical portion, according to some examples.

FIG. 3A is a cross-sectional side view of an elongated tool and an electrically conductive stylet including a cannulated body and a straightening member, according to some examples.

FIG. 3B is a cross-sectional side view of the elongated tool of FIG. 3A with the straightening member withdrawn to form a helical portion of the cannulated body, according to some examples.

FIG. 4 is a cross-sectional side view of an elongated tool and an electrically conductive stylet including a stylet shaft and brush portion, according to some examples.

FIG. 5A is a cross-sectional side view of an elongated tool and an electrically conductive stylet including a stylet shaft and expandable basket portion in an unexpanded configuration, according to some examples.

FIG. 5B is a cross-sectional side view of the elongated tool and the electrically conductive stylet of FIG. 5A with the expandable basket portion in an expanded configuration, according to some examples.

FIG. 6A is a cross-sectional side view of an elongated tool and an electrically conductive stylet including a stylet shaft and plurality of curved tines, according to some examples.

FIG. 6B is a cross-sectional side view of the elongated tool and the electrically conductive stylet of FIG. 6A with the ends of the tines curved away from a distal end of the elongated tool, according to some examples.

FIG. 7A is a cross-sectional side view of an elongated tool and an electrically conductive stylet including a stylet shaft and plurality of tines, according to some examples.

FIG. 7B is a cross-sectional side view of the elongated tool and the electrically conductive stylet of FIG. 7A with the ends of the tines extended distally and away from a central axis of the stylet shaft, according to some examples.

FIG. 8 is a cross-sectional side view of an elongated tool and an electrically conductive stylet with a conductive fluid extending between the tool and the stylet, according to some examples.

FIG. 9 is a cross-sectional side view of a bipolar assembly including an elongated tool with electrically conductive and insulated portions and an electrically conductive stylet engaged with one of the electrically conductive portions of the elongated tool, according to some examples.

FIG. 10 is a cross-sectional side view of a bipolar assembly including an elongated tool with electrically conductive and insulated portions, a first electrically conductive stylet engaged with one of the electrically conductive portion of the elongated tool, and a second electrically conductive grounding stylet engaged with another of the electrically conductive portions of the elongated tool, according to some examples.

FIG. 11 is a flowchart illustrating a method of delivering energy to a target tissue, according to some examples.

FIG. 12 is a flowchart illustrating a method of delivering energy to a target tissue, according to some examples.

FIG. 13 is a simplified diagram of a robot-assisted medical system according to some examples.

FIG. 14A and 14B are simplified diagrams of a medical instrument system according to some examples.

Various examples described herein and their advantages are described in the detailed description that follows. It should be appreciated that like reference numerals are used to identify like elements illustrated in one or more of the figures for purposes of illustrating but not limiting the various examples described herein.

### DETAILED DESCRIPTION

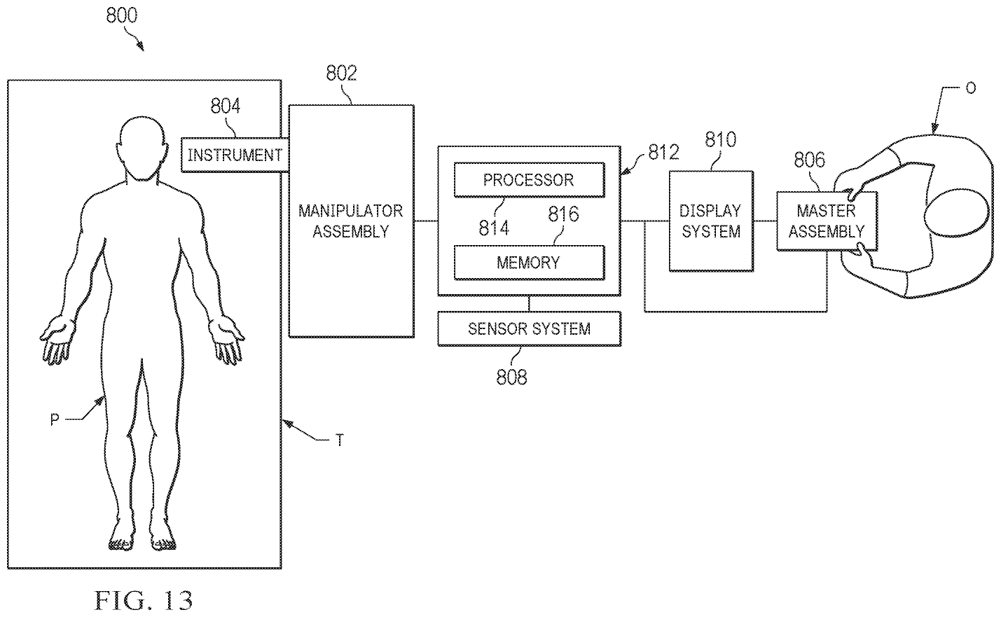

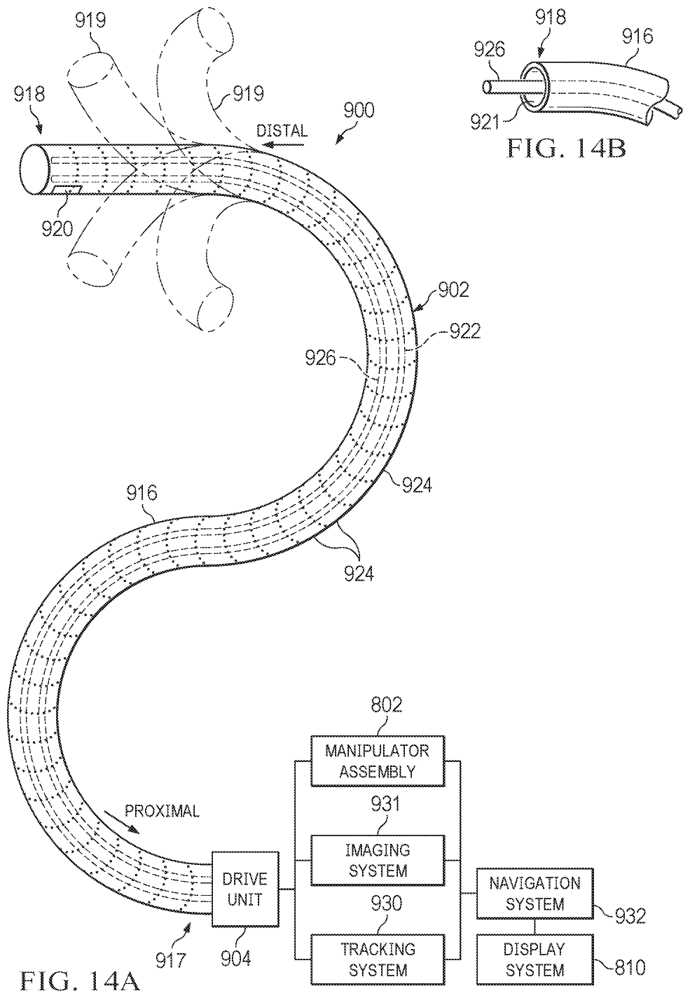

In various examples, an elongated tool, such as a needle, may include a lumen through which one or a series of implements may be passed to conduct therapeutic, diagnostic, or other medical procedures. For example, a biopsy stylet may be inserted through a needle positioned within a target tissue to obtain a tissue sample. The biopsy stylet may be withdrawn and replaced by an electrically conductive stylet that energizes a conductive portion of the needle to deliver energy to the target tissue. For the electrically conductive stylet to deliver predictable and reliable energy to the needle, the stylet may remain in consistent electrical contact with the needle either via direct contact or via contact with a conductive medium. Various examples of needle and stylet configurations are provided to promote or maintain reliable electrical contact between the needle and the electrically conductive stylet. The systems described herein may be used to perform an energized treatment including an ablation or electroporation procedure on the target tissue. An ablation procedure may deliver heat or cold energy to the target tissue, using for example radio frequency (RF) ablation or cryoablation, to burn, scar, or otherwise destroy localized tissue. For example, RF ablation may be performed using a constant energy (current or voltage) to generate thermal effects. An electroporation procedure may use high voltage pulses to create temporary pores in cell membranes through which DNA, a drug, or other substance may be introduced into cells. The pores may be created by destroying or modifying cell walls. The present disclosure describes elongated tools that may be used, for example, in medical systems to provide ablation, electroporation, or other treatments that involve the delivery of energy to target tissue. Examples of medical systems that may incorporate any of the flexible elongate devices described herein are provided at FIGS. 13 and 14.

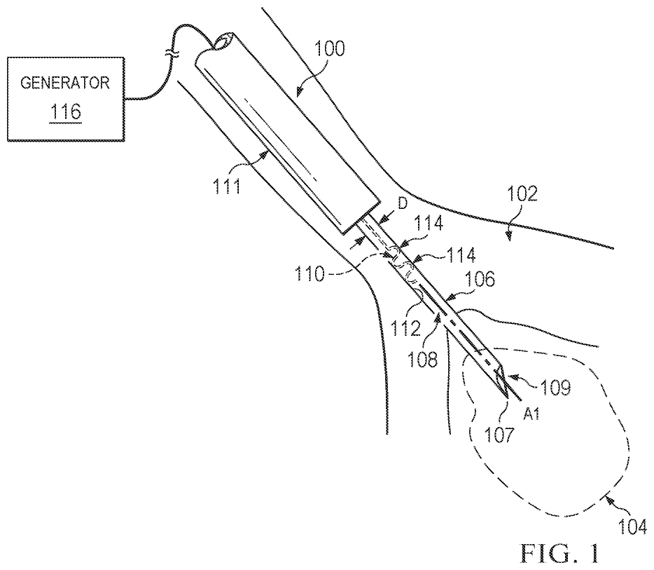

FIG. 1 illustrates a medical instrument system 100 extending with an anatomic passageway 102 and into a target tissue 104. The target tissue may be, for example a tumor, a lymph node, or other tissue to be investigated and/or treated. The medical instrument system 100 may include an elongated tool 106 having a lumen 108 in which extends an electrically conductive stylet 110. Optionally, the medical instrument system 100 may be extended from a delivery device 111, such as delivery catheter, bronchoscope, or other type of delivery device, which may be navigated within the anatomic passageway 102 and parked near the target tissue to create a deployment location for the elongated tool 106. In some examples, the elongated tool 106 may be flexible with an inner wall 112 defining the lumen 108. In some examples, the elongated tool 106 may be a needle including a pointed tip 107 and an aperture 109, the lumen 208 extending to the aperture 109. In some examples, the stylet 110 may be flexible and may contact the inner wall 112 at a plurality of points 114 to transmit electromagnetic energy from the stylet 110 to the tool 106. At least a portion of the stylet 110 may be shaped to extend away from a central axis Al of the lumen 108 and into contact with the inner wall 112. In some examples, portions of the stylet 110 that extend away from the central axis Al and contact the inner wall 122 may also exert a force on the inner wall 112 to enhance the electrical contact between the stylet 110 and the tool 106. In some examples, the configuration of the stylet 110 may be changed from an unexpanded configuration to an expanded configuration, where the diameter D of the stylet 110 is larger in the expanded configuration than the unexpanded configuration. In the unexpanded configuration, the stylet 110 more easily moves within the lumen 108 of the tool 106. In the expanded configuration, portions of the stylet extend away from the central axis Al to contact the inner wall 112, forming the electrical contacts between the stylet 110 and the tool 106. The stylet 110 may exert forces on the inner wall 112 at the plurality of points 114.

For example, the stylet 110 may have a portion shaped as an undulating wave, a coil, a brush, or other configurations that cause the stylet 110 to engage the inner wall 112 at the plurality of points 114. Optionally, the electrically conductive stylet 110 may be coupled to an energy generator 116. The energy generator 116 may be, for example an RF generator or a pressurized gas cryoablation generator for generating heat or cold energy. The energy generator 116 may include components, including hardware, software, and consumable materials, to be used to conduct a variety of ablation or electroporation procedures including pulsed radiofrequency ablation, continuous radiofrequency ablation, water-cooled radio frequency ablation, cryo-neurolysis, cryoablation, microwave ablation, laser ablation, ultrasound ablation, irreversible electroporation, reversible electroporation, or other types of ablation or electroporation. In some examples, electricity delivered by the stylet via the electrical contacts to the tool may cause the tool to emit energy that may be used to perform an energized treatment including an ablation or electroporation procedure on the target tissue. An ablation procedure may deliver heat or cold energy to the target tissue, using for example radio frequency (RF) ablation or cryoablation, to burn, scar, or otherwise destroy localized tissue. An electroporation procedure may use high voltage pulses to create temporary pores in cell membranes through which DNA, a drug, or other substance may be introduced into cells. The pores may be created by destroying or modifying cell walls. In some examples, the stylet 110 may be removable, freeing the lumen 108 to be used for passage of other tools or substances. For example, a biopsy tool or medications may be passed through the lumen 108 while the stylet 110 is removed.

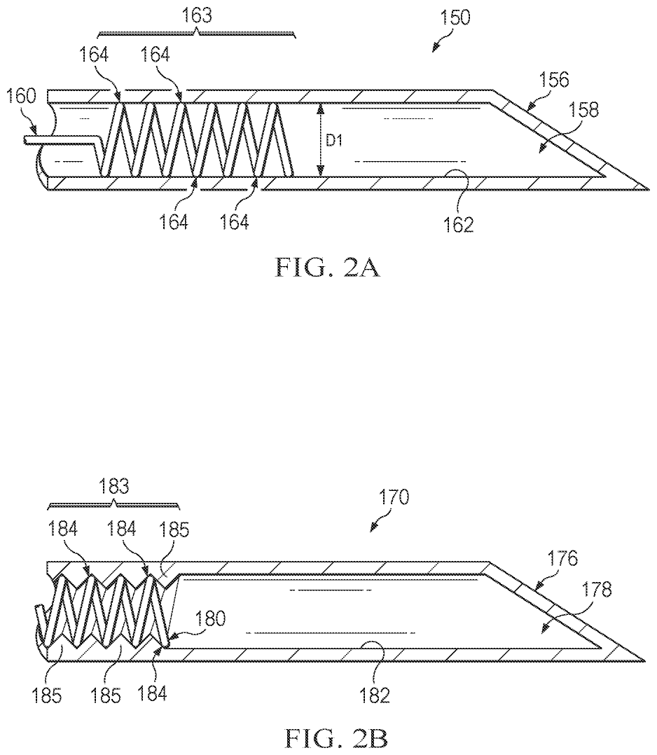

FIG. 2A illustrates a cross-sectional side view of a medical instrument system 150 including an elongated tool 156 (e.g., the elongated tool 106) in which extends an electrically conductive stylet 160 including a helical portion 163. The elongated tool 156 may include a lumen 158 bounded by an inner wall 162. The diameter D1 of the helical portion 163 may be slightly constrained by the inner wall 162 to maintain contact between the helical portion and the inner wall 162. In other words, the diameter D1 of the unconstrained helical portion 163 may be slightly larger than the diameter of the inner wall 162. The helical portion 163 may maintain multiple points of contact 164 with the inner wall 162. The helical portion 163 may exert forces on the inner wall 162 at the points 164. The stylet 160 and the elongated tool 156 may be formed of any of a variety of electrically conductive materials including stainless steel, titanium, titanium coated stainless steel, or a nickel-titanium alloy (e.g., nitinol). The stylet 110 and the elongated tool 106 may be formed of any of a variety of electrically conductive materials including stainless steel, titanium, titanium coated stainless steel, or a nickel-titanium alloy (e.g., nitinol). In some examples, a plating material may be applied to the stylet or elongated tool to provide or improve electrical conductivity. For example, a base material such as stainless steel or nitinol that may have the desired mechanical properties (e.g., durability, strength, elasticity) may be plated with a material such as gold that has superior electrical properties to the base material.

The helical portion 163 may have a helix or otherwise spiral shape and may be referred to as a pigtail, corkscrew, coil-spring or other similar shape. In some examples, the shape of the helical portion may be thermally responsive. For example, the portion 163 may be formed of a nitinol material that is preset to assume the helical shape in response to heat energy. In such an example, the nitinol portion may have a non-helical shape in the absence of an applied electrical current, but when an electrical current is applied, the heat energy may cause a modification of the shape and induce the formation of the predetermined helical configuration.

FIG. 2B illustrates a cross-sectional side view of a medical instrument system 170 including an elongated tool 176 (e.g., an elongated tool 106) in which extends an electrically conductive stylet 180 including a helical portion 183. The elongated tool 176 may include a lumen 178 bounded by an inner wall 182. In this example the inner wall 182 may include threads 185. The helical portion 183 of the stylet 180 may be threadedly engaged with the inner wall 182 such that the helical portion 183 nests between the threads 185. The threads 185 allow the helical portion 183 to maintain multiple points of contact 184 with the threads 185 and/or the inner wall 182 of the tool 176 and can help ensure more reliable and persistent contacts. The helical portion 183 may exert forces on the inner wall and threads at the plurality of points 184.

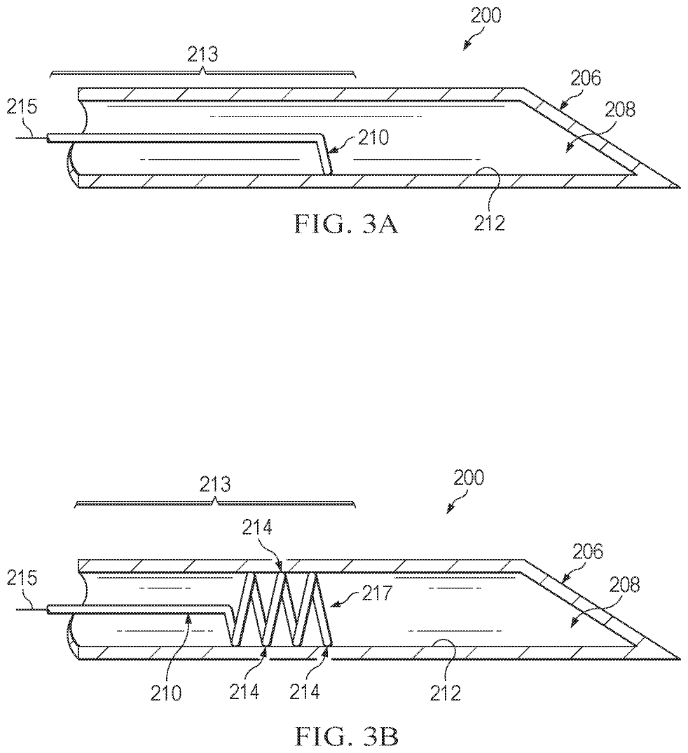

FIG. 3A illustrates a cross-sectional side view of a medical instrument system 200 including an elongated tool 206 (e.g., an elongated tool 106) in which extends an electrically conductive stylet 210. The elongated tool 206 may include a lumen 208 bounded by an inner wall 212. In this example, the stylet 210 includes a cannulated portion 213 with an elongated passage that has a preformed helical shape and includes a straightening member 215 that extends within the cannulated portion 213 to cause the portion 213 to assume a straightened configuration. The stylet 210 may be inserted into the lumen 208 with the straightening member 215 extended in the cannulated portion 213 causing the stylet to have low profile, straightened configuration. With the stylet 210 longitudinally in place within the lumen 208, the straightening member 215 may be withdrawn from the cannulated portion 213. Without the straightening member 215 extending in the cannulated portion 213, the cannulated portion 213 may revert to an expanded configuration having a (e.g., preformed or preset) helical shape 217, as shown in FIG. 3B. In some examples, the straightening member 215 may be a formed of a material that is more rigid than the cannulated portion 213 of the stylet 210. In some examples, the cannulated portion 213 may be formed from nitinol or another shape-memory material. With the straightening member 215 withdrawn, the diameter of the cannulated portion 213 may be slightly constrained by the inner wall 212 to maintain contact between the helical cannulated portion 213 and the inner wall 212. In the expanded configuration, the portion 213 may maintain multiple points of contact 214 with the inner wall 212. The stylet 210 may exert forces on the inner wall 212 at the plurality of points 214.

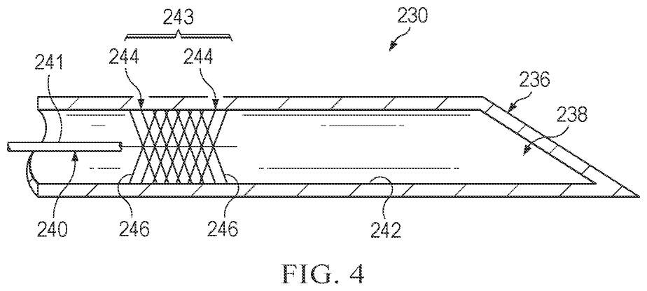

FIG. 4 illustrates a cross-sectional side view of a medical instrument system 230 including an elongated tool 236 (e.g., an elongated tool 106) in which extends an electrically conductive stylet 240. The elongated tool 236 may include a lumen 238 bounded by an inner wall 242. In this example, the stylet 240 may include a flexible shaft 241 and a brush portion 243 that includes a plurality of bristles 246 that extend into multiple points of contact 244 with the inner wall 242. In some examples, the flexible shaft 241 may be cannulated such that brush portion 243 may be retracted into the flexible shaft 241 to create low-profile configuration of the stylet and may be extended from the flexible shaft to create an expanded configuration in which the bristles engage the inner wall 242. The brush portion 243 may exert forces on the inner wall 242 at the plurality of points 244. In other examples, the brush portion 243 may be fixed to a distal end of the shaft 241.

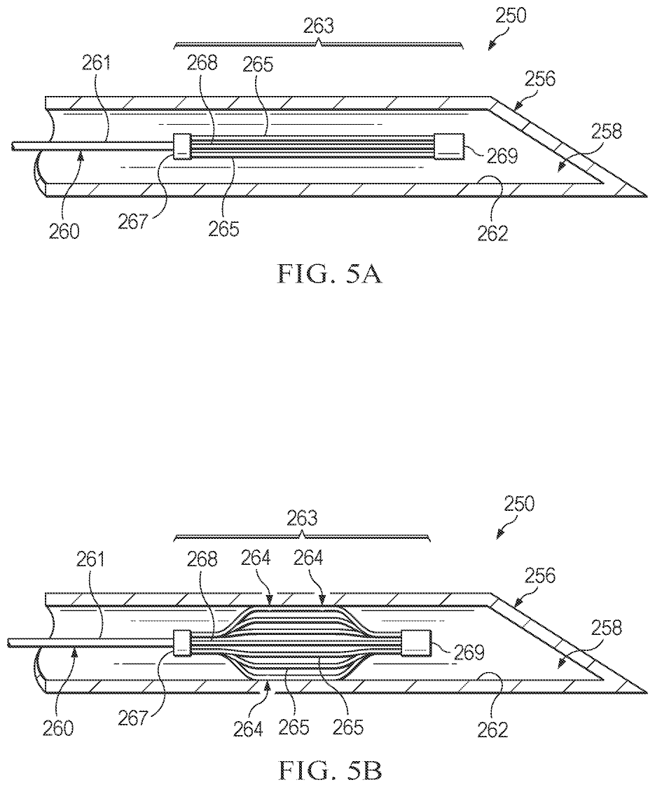

FIG. 5A illustrates a cross-sectional side view of a medical instrument system 250 including an elongated tool 256 (e.g., an elongated tool 106) in which extends an electrically conductive stylet 260. The elongated tool 256 may include a lumen 258 bounded by an inner wall 262. In this example, the stylet 260 may include a flexible shaft 261 and a basket portion 263 that includes a plurality of splines 265. The basket portion 263 may be inserted into the lumen 258 in a collapsed, unexpanded configuration (also referred to as a low-profile configuration) as shown in FIG. 5A and may be adjusted to an expanded configuration as shown in FIG. 5B. In the expanded configuration, the splines 265 extend radially into multiple points of contact 264 with the inner wall 262. In some examples, the basket portion 263 may be formed from a nitinol tube with slits cut along the longitudinal dimension of the tube to create the splines in the remaining tube. In other examples, the splines may include an array of bundled wires. In some examples, the basket portion 263 includes a proximal cap 267 at a proximal end of the splines 265 and a distal cap 269 at a distal end of the splines 265. The caps 267, 269 may be integrally formed with the splines (e.g., an uncut portion of an original nitinol tube) or may be attached by an adhesive, welding, or other coupling technique. In some examples, an actuator 268 may be coupled to one or both of the caps 267, 269 to transition the basket portion 263 between the collapsed and the expanded configuration. For example, as shown in FIG. 5B, the actuator 268 may be coupled to the distal cap 269. Pulling the actuator 268, with the proximal cap 267 held stationary may move the distal cap 269 toward the proximal cap 267, causing the splines to bow outward and into the expanded configuration in which the splines engage the inner wall 262. In another example, the actuator may be coupled to the proximal cap 267. Pushing the actuator, with the distal cap 269 held stationary may move the proximal cap 267 toward the distal cap 269, causing the splines to bow outward and into the expanded configuration in which the splines engage the inner wall 262. The splines 265 may exert forces on the inner wall 262 at the plurality of points 264.

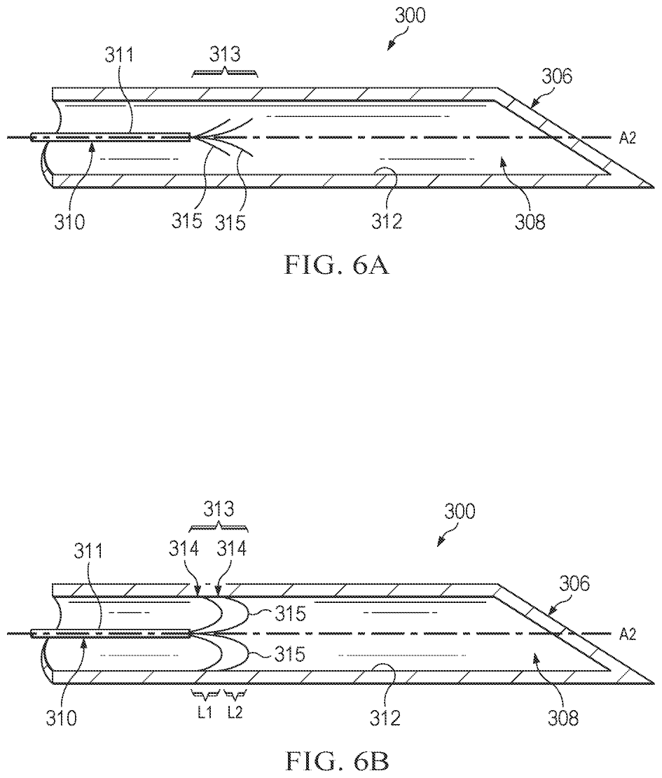

FIG. 6A illustrates a cross-sectional side view of a medical instrument system 300 including an elongated tool 306 (e.g., an elongated tool 106) in which extends an electrically conductive stylet 310. The elongated tool 306 may include a lumen 308 bounded by an inner wall 312. In this example, the stylet 310 may include a flexible shaft 311 and an expandable portion 313 that includes a plurality of curved or arc-shaped tines 315. The expandable portion 313 may be inserted into the lumen 308 in a collapsed, low-profile configuration as shown in FIG. 6A and may be adjusted to an expanded, umbrella-shaped configuration as shown in FIG. 6B. In some examples, the flexible shaft 311 may be cannulated and sized to receive the tines 315 when the expandable portion 313 is in the collapsed configuration. In the collapsed configuration, the cannulated shaft 311 may extend over the length of or a partial length of the expandable portion 313 to straighten or partially straighten the tines 315 into the low-profile configuration. In the expanded configuration, as shown in FIG. 6B, the tines 315 may extend distally of the cannulated shaft 311 and may arc away from a longitudinal axis A2 of the shaft 311 forming the umbrella-shaped expandable portion 313. In the expanded configuration, the tines 315 may extend into multiple points of contact 314 with the inner wall 312. The tines 315 may exert forces on the inner wall 312 at the plurality of points 314. The arc-shaped tines 315 may have ends that bend outward and back toward the shaft 311. The tines 315 may be formed of a shape-memory material such as nitinol and may have a preset arc shape. In some examples the flexible shaft 311 may be moved distally to bend the tines 315 into the straightened, collapsed configuration and may be moved proximally to remove constraint on the tines 315, allowing them to curl into the expanded configuration. Alternatively, the tines 315 may be moved proximally to draw them into the flexible shaft 311 into the collapsed configuration and may be moved distally relative to the flexible shaft 311 to form the expanded configuration. In some examples, an actuator (e.g., a push/pull wire) may move the flexible shaft and/or the tines 315. For example, the actuator may be pushed to advance the tines 315 distally relative to the cannulated shaft 311, allowing the pre-bent tines to curl into the expanded configuration. In some examples, an actuator (e.g., a push wire) may be pushed to advance the tines through openings or apertures in the cannulated shaft, allowing the pre-bent tines to curl into the expanded configuration. In some examples, an opposite motion (e.g., a proximal motion) of the actuator may retract the tines 315 into the cannulated shaft and into the collapsed configuration. In some examples the tines may be configured in layers along the axis A2. For example, FIG. 6B illustrates an expandable portion 313 with tines 315 in a layer L1 and a layer L2.

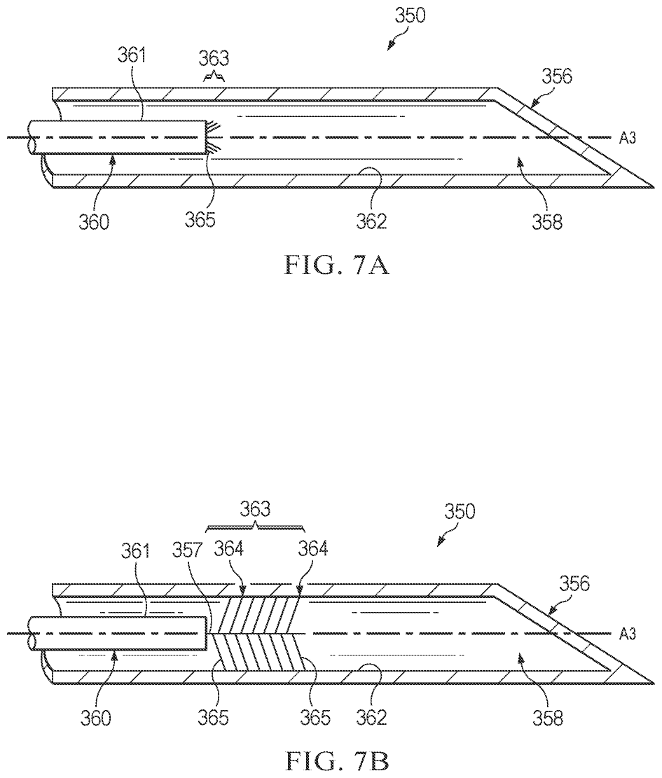

FIG. 7A illustrates a cross-sectional side view of a medical instrument system 350 including an elongated tool 356 (e.g., an elongated tool 106) in which extends an electrically conductive stylet 360. The elongated tool 356 may include a lumen 358 bounded by an inner wall 362. In this example, the stylet 360 may include a flexible shaft 361 and an expandable portion 363 that includes a plurality of straight, outwardly and distally extending tines 365. The expandable portion 363 may be inserted into the lumen 358 in a collapsed, low-profile configuration as shown in FIG. 7A and may be adjusted to an expanded configuration as shown in FIG. 7B. In some examples, the flexible shaft 361 may be cannulated and sized to receive the tines 365 when the expandable portion 363 is in the collapsed configuration. In the collapsed configuration, the cannulated shaft 361 may extend over the length of or a partial length of the expandable portion 363 to bend the tines 315 toward the central axis A3 and into the low-profile configuration. In the expanded configuration, as shown in FIG. 7B, the tines 365 may extend distally of the cannulated shaft 361 and may outward at an angle (less than or approximately 90 degrees) from the longitudinal axis A3 of the shaft 361. In the expanded configuration, the tines 365 may extend into multiple points of contact 364 with the inner wall 362. The tines 365 may exert forces on the inner wall 362 at the plurality of points 364. In this example, the tines 315 may be generally straight. In some examples the flexible shaft 361 may be moved distally to bend the tines 365 into the straightened, collapsed configuration and may be moved proximally to remove constraint on the tines 365, allowing them to flare or splay into the expanded configuration. Alternatively, the tines 365 may be moved proximally to draw them into the flexible shaft 361 into the collapsed configuration and may be moved distally relative to the flexible shaft 361 to form the expanded configuration. In some examples, an actuator 357 (e.g., a push/pull wire) may move the flexible shaft and/or the tines. For example, the actuator pushed to advance the tines 365 distally relative to the cannulated shaft 361, allowing them to flare or splay into the expanded configuration. In some examples, an actuator (e.g., a push/pull wire) may be pushed to advance the tines through openings or apertures in the cannulated shaft, allowing the tines to flare or splay into the expanded configuration. In some examples, an opposite motion (e.g., a proximal motion) of the actuator may retract the tines 365 into the cannulated shaft and into the collapsed configuration.

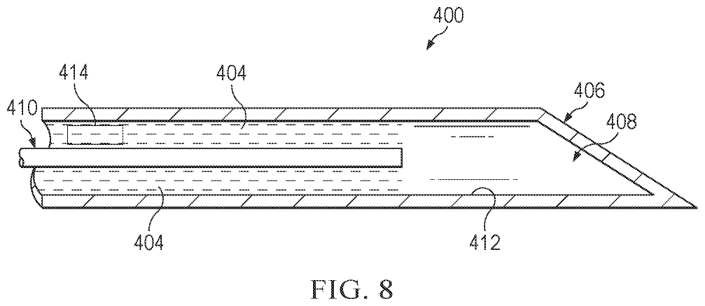

FIG. 8 illustrates a cross-sectional side view of a medical instrument system 400 including an elongated tool 406 (e.g., an elongated tool 106) and an electrically conductive stylet 410 with a conductive fluid 404 extending between the tool 406 and the stylet 410. The elongated tool 406 may include a lumen 408 bounded by an inner wall 412. In this example, the stylet 410 may not directly contact the inner wall 412 but may be electrically coupled to the inner wall 412 by the fluid 404. In some examples, the fluid 404 may be a saline solution. Energy from the stylet 410 may flow through the fluid 404 to energize the tool 406. Optionally, a sensor 414 may measure properties of the fluid including, for example, a temperature or a flow rate. In some examples, the fluid may enter the tool through an injection port (not shown) and a drip rate of fluid 404 into the tool 406 may be adjustable. In various examples, the fluid 404 may stay within the lumen 408, may flow from a distal end of the tool (e.g., through the aperture 109), or may be evacuated through the injection port.

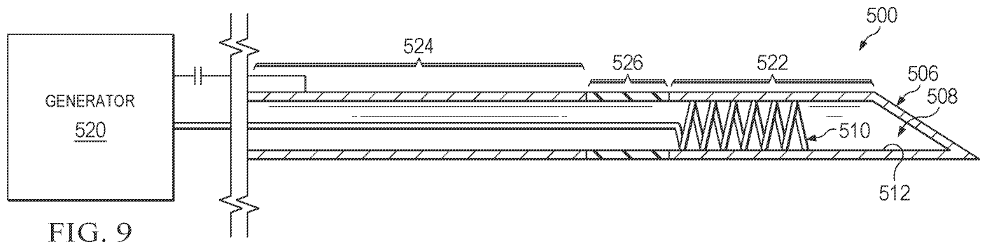

The examples of FIGS. 1-8 may be monopolar energy delivery assemblies with a grounding electrode placed apart from the electrically conductive tool, on or in the patient anatomy. Alternatively, the examples may be configured as bipolar energy delivery assemblies. FIG. 9 is a cross-sectional side view of a medical instrument system 500 with a bipolar energy delivery assembly including an elongated tool 506 in which extends an electrically conductive stylet 510. The elongated tool 506 may include a lumen 508 bounded by an inner wall 512. In this example, the stylet 510 may be configured similarly to stylet 160, but any of the stylet examples of FIGS. 1-8 may be suitable for use in a bipolar energy delivery assembly. In the example of FIG. 9, the stylet 510 may be electrically connected to an energy generator 520 (e.g., energy generator 116) which in this example may be an RF generator. The elongated tool 506 may include an electrically conductive segment 522 and an electrically conductive segment 524 separated by an insulated segment 526. In this example, an electrical current may flow from the generator 520 to the stylet 510. As previously described, the stylet 510 may be engaged with the inner wall 512 of the electrically conductive segment 522 of elongated tool 506 to transmit the electrical current to the tool 506 and into the target tissue. The electrically conductive segment 524 may serve as a grounding electrode for the electrical current through the tool 556.

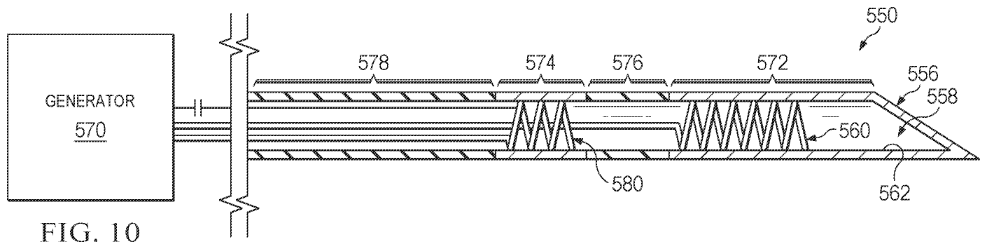

FIG. 10 is a cross-sectional side view of a medical instrument system 550 with a bipolar energy delivery assembly including an elongated tool 556 in which extends an electrically conductive stylet 560. The elongated tool 556 may include a lumen 558 bounded by an inner wall 562. In this example, the stylet 560 may be configured similarly to stylet 160, but any of the stylet examples of FIGS. 1-7B may be suitable for use in a bipolar energy delivery assembly. In the example of FIG. 10, the stylet 560 may be electrically connected to an energy generator 570 (e.g., energy generator 116) which in this example may be an RF generator. The elongated tool 556 may include an electrically conductive segment 572 and an electrically conductive segment 574 separated by an insulated segment 576. In this example, the tool 556 proximal of the conductive segment 574 may include an insulated segment 578. The stylet 560 may be introduced into the tool 556 and may engage with the inner wall 562 of the electrically conductive segment 572 of elongated tool 556 to transmit the electrical current to the tool 556 and into the target tissue. A second electrically conductive grounding stylet 580 may engage the wall 562 at the electrically conductive segment 574. The electrically conductive segment 574 may thus serve as a grounding electrode, coupled to the grounding stylet 580 for grounding the electrical current from the target tissue through the tool 556. The grounding stylet 580 may be electrically insulated from the stylet 560 within the lumen 558. For example the grounding stylet 580 and the stylet 560 maybe insulated and bundled together with both extending to the generator 520

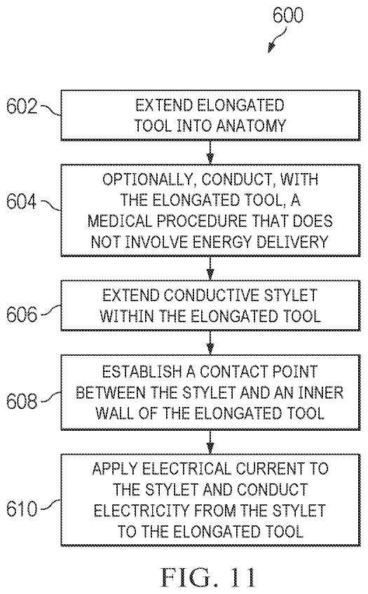

FIG. 11 is a flowchart illustrating a method 600 for delivering energy to a target tissue. The methods described herein are illustrated as a set of operations or processes and are described with continuing reference to additional figures. Not all of the illustrated processes may be performed in all embodiments of the methods. Additionally, one or more processes that are not expressly illustrated in may be included before, after, in between, or as part of the illustrated processes. In some embodiments, one or more of the illustrated processes may be omitted. In some embodiments, one or more of the processes may be implemented, at least in part, in the form of executable code stored on non-transitory, tangible, machine-readable media that when run by one or more processors (e.g., the processing units of a control system, such as control system 812) may cause the one or more processors to perform one or more of the processes. In one or more embodiments, the processes may be performed by a control system.

At a process 602 of the method 600, an elongated tool may be extended into a patient anatomy. For example, as shown in FIG. 1, the elongated tool 106 may be a needle extended into the anatomic passageway 102 of a patient and into the target tissue 104. Optionally, the elongated tool may be delivered to a deployment location by a delivery device (e.g., delivery device 111). The tool may be extended from the delivery device and/or the delivery device may be retracted to expose the tool.

At an optional process 604, a medical procedure, such as a biopsy or other procedure that does not involve energy delivery, may be performed with the elongated tool. For example, the elongated tool (e.g., tool 106) may be a cannulated needle that may also be used to perform a biopsy, prior to an energy delivery procedure such as ablation or electroporation. The needle may be delivered into the target tissue and may first be used alone or with a non-energized stylet to sample tissue in a biopsy procedure. If the biopsy procedure confirms that the target tissue is diseased or otherwise may benefit from energy therapy, the processes 606-610 may be conducted to treat the tissue. For example, an electrode stylet may be inserted within the needle lumen and energized. The current from the electrode stylet may activate the needle to deliver energy to the target tissue and provide a means to treat the target tissue as described. Thus, the cannulated needle may serve as a tool to reach target tissue to conduct multiple procedures, including a biopsy procedure in which the lumen of the needle is used to receive tissue during a biopsy and an energy delivery procedure in which the wall surrounding the lumen of the needle provides a surface to create electrical contact points with the electrode stylet during an electroporation or ablation procedure. Using the same tool for biopsy and for energy therapy may allow for a more efficient and shorter duration medical treatment, without the need to deploy multiple tools.

At a process 606, a conductive stylet may be extended within the elongated tool. For example, the electrically conductive stylet 110 may be extended into the elongated tool 106 in an unexpanded configuration. In other examples the conductive stylet may include any other stylet configurations (e.g., stylets 160, 180, 210, 240, 260, 310, 360, 410, 510, 560) described herein. In some examples, the conductive stylet may be within the elongated tool when the elongated tool is extended into the patient anatomy.

At a process 608, one or a plurality of contact points may be established between the conductive stylet and an inner wall of the elongated tool. For example, the electrically conductive stylet 110 may engage the tool 106 at one or multiple contact points 114. In some examples, one or more portions of the conductive stylet extends away from a central axis Al of the lumen of the elongated tool to establish the contact points. In some examples, establishing the contact points may include changing a configuration of the conductive stylet, such as from an unexpanded configuration to an expanded configuration.

At a process 610, an electrical current may be applied to the stylet and the current may be conducted from the stylet to the elongated tool via the contact points. For example, an electrical current may be conducted from the electrically conductive stylet 110 to the electrically conductive tool for ablation or electroporation of the target tissue.

Optionally, the method may continue by removing the conductive stylet from the elongated tool. The elongated tool either alone or with a replacement tool extended in the tool lumen may be used to conduct additional procedures, such as biopsy procedures, on the target tissue. For example, the conductive stylet 110 may be removed from lumen 108 of the tool 106. The lumen 108 may then be used to sample tissue from the target tissue 104 to conduct a biopsy analysis.

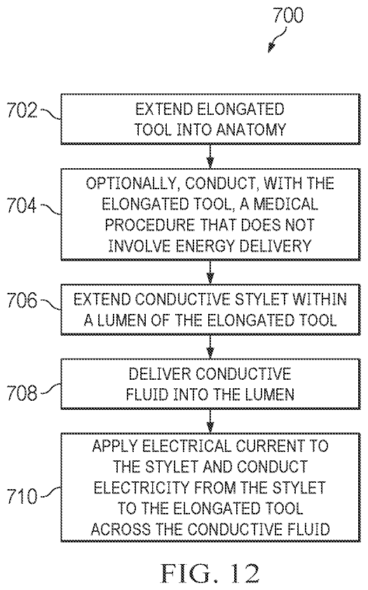

FIG. 12 is a flowchart illustrating a method 700 for delivering energy to a target tissue. At a process 702, an elongated tool may be extended into a patient anatomy. For example, as shown in FIG. 8, the elongated tool 406 may be a needle that may be extended into an anatomic passageway of a patient and into nearby target tissue. Optionally, the elongated tool 406 may be delivered to a deployment location by a delivery device (e.g., delivery device 111) and the tool 406 may be extended from the delivery device.

At an optional process 704, a medical procedure, such as a biopsy or other procedure that does not involve energy delivery, may be performed with the elongated tool. For example, the elongated tool (e.g., tool 106) may be a cannulated needle that may also be used to perform a biopsy, prior to an energy delivery procedure such as ablation or electroporation. The needle may be delivered into the target tissue and may first be used alone or with a non-energized stylet to sample tissue in a biopsy procedure. If the biopsy procedure confirms that the target tissue is diseased or otherwise may benefit from energy therapy, the processes 706-710 may be conducted to treat the tissue. For example, an electrode stylet may be inserted within the needle lumen and energized. The current from the electrode stylet may activate the needle to deliver energy to the target tissue and provide a means to treat the target tissue as described. Thus, the cannulated needle may serve as a tool to reach target tissue to conduct multiple procedures, including a biopsy procedure in which the lumen of the needle is used to receive tissue during a biopsy and an energy delivery procedure in which the wall surrounding the lumen of the needle provides a surface to create electrical contact points with the electrode stylet during an electroporation or ablation procedure. Using the same tool for biopsy and for energy therapy may allow for a more efficient and shorter duration medical treatment, without the need to deploy multiple tools.

At a process 706, a conductive stylet may be extended within a lumen of the elongated tool. For example, the electrically conductive stylet 410 may be extended into the lumen 408 of the elongated tool 406. In other examples the conductive stylet may include any other stylet configurations described herein, including stylet configurations that engage the inner wall of the tool at one or more locations.

At a process 708, a conductive fluid may be introduced into the lumen 408. For example, a conductive fluid 404, such as saline, may be introduced into the lumen 408 and may extend between the stylet 410 and the inner wall 412. In some examples the sensor 414 may monitor the temperature, flow rate, or other parameters associated with the conductive fluid.

At a process 710, an electrical current may be applied to the stylet and the current may be conducted from the stylet to the elongated tool, across the conductive fluid. For example, an electrical current may be conducted from the electrically conductive stylet 410 to the electrically conductive tool 406 through the conductive fluid 404. The energized tool 406 may be used for ablation or electroporation of the target tissue.

Optionally, the method may continue by removing the conductive stylet from the elongated tool. The elongated tool either alone or with a replacement tool extended in the tool lumen may be used to conduct additional procedures, such as biopsy procedures, on the target tissue. For example, the conductive stylet 410 may be removed from lumen 408 of the tool 406. The lumen 408 may then be used to sample tissue from the target tissue to conduct a biopsy analysis.

In some examples, medical procedure may be performed using hand-held or otherwise manually controlled systems and tools of this disclosure. In other examples, the described imaging probes and tools many be manipulated with a robot-assisted medical system as shown in FIG. 13. FIG. 13 illustrates a robot-assisted medical system 800. The robot-assisted medical system 800 generally includes a manipulator assembly 802 for operating a medical instrument system 804 (including, for example, medical instrument system 100 or any of the medical instrument systems described herein) in performing various procedures on a patient P positioned on a table T in a surgical environment 801. The manipulator assembly 802 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. A master assembly 806, which may be inside or outside of the surgical environment 801, generally includes one or more control devices for controlling manipulator assembly 802. Manipulator assembly 802 supports medical instrument system 804 and may include a plurality of actuators or motors that drive inputs on medical instrument system 804 in response to commands from a control system 812. The actuators may include drive systems that when coupled to medical instrument system 804 may advance medical instrument system 804 into a naturally or surgically created anatomic orifice. Other drive systems may move the distal end of medical instrument system 804 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulatable end effector of medical instrument system 804 for grasping tissue in the jaws of a biopsy device and/or the like.

Robot-assisted medical system 800 also includes a display system 810 for displaying an image or representation of the surgical site and medical instrument system 804 generated by a sensor system 808, which may include an endoscopic imaging system. Display system 810 and master assembly 806 may be oriented so operator O can control medical instrument system 804 and master assembly 806 with the perception of telepresence.

In some examples, medical instrument system 804 may include components for use in surgery, biopsy, ablation, illumination, irrigation, or suction. Medical instrument system 804, together with sensor system 808 may be used to gather (i.e., measure) a set of data points corresponding to locations within anatomic passageways of a patient, such as patient P. In some examples, medical instrument system 804 may include components of the endoscopic imaging system, which may include an imaging scope assembly or imaging that records a concurrent or real-time image of a surgical site and provides the image to the operator or operator O through the display system 810. The concurrent image may be, for example, a two or three-dimensional image captured by an imaging instrument positioned within the surgical site. In some examples, the endoscopic imaging system components may be integrally or removably coupled to medical instrument system 804. However, in some examples, a separate endoscope, attached to a separate manipulator assembly may be used with medical instrument system 804 to image the surgical site. The endoscopic imaging system may be implemented as hardware, firmware, software, or a combination thereof which interact with or are otherwise executed by one or more computer processors, which may include the processors of the control system 812.

The sensor system 808 may include a position/location sensor system (e.g., an electromagnetic (EM) sensor system) and/or a shape sensor system for determining the position, orientation, speed, velocity, pose, and/or shape of the medical instrument system 804.

Robot-assisted medical system 800 may also include control system 812. Control system 812 includes at least one memory 816 and at least one computer processor 814 for effecting control between medical instrument system 804, master assembly 806, sensor system 808 (including endoscopic imaging system), intra-operative imaging system 818, and display system 810. Control system 812 also includes programmed instructions (e.g., a non-transitory machine-readable medium storing the instructions) to implement some or all of the methods described in accordance with aspects disclosed herein, including instructions for providing information to display system 810.

Control system 812 may further include a virtual visualization system to provide navigation assistance to operator O when controlling medical instrument system 804 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

FIG. 14A is a simplified diagram of a medical instrument system 900 configured in accordance with various embodiments of the present technology. The medical instrument system 900 includes an elongate flexible device 902 (e.g., delivery device 111), such as a flexible catheter, coupled to a drive unit 904. The elongate flexible device 902 includes a flexible body 916 having a proximal end 917 and a distal end or tip portion 918. The medical instrument system 900 further includes a tracking system 930 for determining the position, orientation, speed, velocity, pose, and/or shape of the distal end 918 and/or of one or more segments 924 along the flexible body 916 using one or more sensors and/or imaging devices as described in further detail below.

The tracking system 930 may optionally track the distal end 918 and/or one or more of the segments 924 using a shape sensor 922. The shape sensor 922 may optionally include an optical fiber aligned with the flexible body 916 (e.g., provided within an interior channel (not shown) or mounted externally). The optical fiber of the shape sensor 922 forms a fiber optic bend sensor for determining the shape of the flexible body 916. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. Pat. No. 7,781,724 (filed Sep. 26, 2006, disclosing “Fiber optic position and shape sensing device and method relating thereto”; U.S. Pat. No. 7,772,541, filed Mar. 12, 2008, titled “ Fiber Optic Position and/or Shape Sensing Based on Rayleigh Scatter”; and U.S. Pat. No. 6,389,187, filed Apr. 21, 2000, disclosing “Optical Fiber Bend Sensor,” which are all incorporated by reference herein in their entireties. In some embodiments, the tracking system 930 may optionally and/or additionally track the distal end 918 using a position sensor system 920. The position sensor system 920 may be a component of an EM sensor system with the position sensor system 920 including one or more conductive coils that may be subjected to an externally generated electromagnetic field. In some embodiments, the position sensor system 920 may be configured and positioned to measure six degrees of freedom (e.g., three position coordinates X, Y, and Z and three orientation angles indicating pitch, yaw, and roll of a base point) or five degrees of freedom (e.g., three position coordinates X, Y, and Z and two orientation angles indicating pitch and yaw of a base point). Further description of a position sensor system is provided in U.S. Pat. No. 6,380,732, filed Aug. 9, 1999, disclosing “Six-Degree of Freedom Tracking System Having a Passive Transponder on the Object Being Tracked,” which is incorporated by reference herein in its entirety. In some embodiments, an optical fiber sensor may be used to measure temperature or force. In some embodiments, a temperature sensor, a force sensor, an impedance sensor, or other types of sensors may be included within the flexible body. In various embodiments, one or more position sensors (e.g., fiber shape sensors, EM sensors, and/or the like) may be integrated within the medical instrument 926 and used to track the position, orientation, speed, velocity, pose, and/or shape of a distal end or portion of medical instrument 926 using the tracking system 930.

The flexible body 916 includes a channel 921 sized and shaped to receive a medical instrument 926 (e.g., elongated tool 106). FIG. 14B, for example, is a simplified diagram of the flexible body 916 with the medical instrument 926 extended according to some embodiments. In some embodiments, the medical instrument 926 may be used for procedures such as imaging, visualization, surgery, biopsy, ablation, illumination, irrigation, and/or suction. The medical instrument 926 can be deployed through the channel 921 of the flexible body 916 and used at a target location within the anatomy. The medical instrument 926 may include, for example, image capture probes, biopsy instruments, ablation needles, electroporation needles, laser ablation fibers, and/or other surgical, diagnostic, or therapeutic tools, including any of the instrument systems described above. The medical instrument 926 may be used with an imaging instrument (e.g., an image capture probe) within the flexible body 916. The imaging instrument may include a cable coupled to the camera for transmitting the captured image data. In some embodiments, the imaging instrument may be a fiber- optic bundle, such as a fiberscope, that couples to an image processing system 931. The imaging instrument may be single or multi-spectral, for example capturing image data in one or more of the visible, infrared, and/or ultraviolet spectrums. The medical instrument 926 may be advanced from the opening of channel 921 to perform the procedure and then be retracted back into the channel 921 when the procedure is complete. The medical instrument 926 may be removed from the proximal end 917 of the flexible body 916 or from another optional instrument port (not shown) along the flexible body 916.

The flexible body 916 may also house cables, linkages, or other steering controls (not shown) that extend between the drive unit 904 and the distal end 918 to controllably bend the distal end 918 as shown, for example, by broken dashed line depictions 919 of the distal end 918. In some embodiments, at least four cables are used to provide independent “up-down” steering to control a pitch of the distal end 918 and “left-right” steering to control a yaw of the distal end 918. Steerable elongate flexible devices are described in detail in U.S. Pat. No. 9,452,276, filed Oct. 14, 2011, disclosing “Catheter with Removable Vision Probe,” and which is incorporated by reference herein in its entirety. In various embodiments, medical instrument 926 may be coupled to drive unit 904 or a separate second drive unit (not shown) and be controllably or robotically bendable using steering controls.

The information from the tracking system 930 may be sent to a navigation system 932 where it is combined with information from the image processing system 931 and/or the preoperatively obtained models to provide the operator with real-time position information. In some embodiments, the real-time position information may be displayed on the display system 810 of FIG. 13 for use in the control of the medical instrument system 900. In some embodiments, the control system 812 of FIG. 13 may utilize the position information as feedback for positioning the medical instrument system 900. Various systems for using fiber optic sensors to register and display a surgical instrument with surgical images are provided in U.S. Pat. No. 8,900,131, filed May 13, 2011, disclosing “Medical System Providing Dynamic Registration of a Model of an Anatomic Structure for Image-Guided Surgery,” which is incorporated by reference herein in its entirety.

In some embodiments, the medical instrument system 900 may be teleoperated or robot-assisted within the medical system 800 of FIG. 13. In some embodiments, the manipulator assembly 802 of FIG. 13 may be replaced by direct operator control. In some embodiments, the direct operator control may include various handles and operator interfaces for hand-held operation of the instrument.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 812) or may be implemented, at least in part, in the form of executable code stored on non-transitory, tangible, machine-readable media that when run by one or more processors (e.g., the processors 814 of control system 812) may cause the one or more processors to perform one or more of the processes.

One or more elements in examples of this disclosure may be implemented in software to execute on a processor of a computer system such as control processing system. When implemented in software, the elements of the examples of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one example, the control system supports wireless communication protocols such as Bluetooth, IrDA, HomeRF, IEEE 802.11, DECT, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

The singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. Components described as coupled may be directly or indirectly communicatively coupled. The auxiliary verb “may” likewise implies that a feature, step, operation, element, or component is optional.

While certain exemplary examples of the invention have been described and shown in the accompanying drawings, it is to be understood that such examples are merely illustrative of and not restrictive on the broad invention, and that the examples of the invention not be limited to the specific constructions and arrangements shown and described, since various other modifications may occur to those ordinarily skilled in the art.

1. A medical system comprising:
a flexible elongated tool in which a lumen extends, the lumen defined by an inner wall of the elongated tool; and
a flexible stylet configured to extend within the lumen and contact the inner wall of the elongated tool at a plurality of points, wherein energy conducted through the stylet is transmitted at the plurality of points to the elongated tool.

2. The medical system of claim 1 wherein at least a portion of the flexible stylet extends away from a central axis of the lumen.

3. The medical system of claim 1 wherein the flexible stylet has an unexpanded configuration and expanded configuration, a diameter of the stylet being larger in the expanded configuration than the unexpanded configuration, and wherein the flexible stylet contacts the plurality of points in the expanded configuration.

4. The medical system of claim 1 wherein the flexible stylet exerts forces on the inner wall at the plurality of points.

5. The medical system of claim 1 wherein the flexible stylet includes a stylet shaft that has a helical shape with an outer diameter larger than an inner diameter of the tool when outside of the lumen.

6. The medical system of claim 1 wherein the flexible stylet includes a stylet shaft that has a helical shape configured to engage threads along the inner wall of the tool.

7. The medical system of claim 1 wherein the elongated tool includes a needle.

8. The medical system of claim 1, further comprising a straightening member configured to extend within an elongated passage in the stylet, wherein withdrawing the straightening member from a portion of the stylet causes the portion of the stylet to revert to a preformed shape.

9. The medical system of claim 8 wherein the preformed shape is a helical shape.

10. (canceled)

11. The medical system of claim 1 where the stylet includes a brush portion with multiple bristles that extend from a shaft of the stylet into contact with the inner wall of the tool.

12. The medical system of claim 1 wherein the stylet includes an expandable basket portion that can be inserted into the tool in a low profile configuration and transitioned to an expanded profile configuration wherein a plurality of splines of the expandable basket portion contact the inner wall of the tool.

13. The medical system of claim 12, wherein the splines are separated by slits cut in a nitinol tube.

14. The medical system of claim 12, wherein the splines include an array of wires.

15. The medical system of claim 12, further comprising an actuator coupled to a cap on the expandable basket portion, wherein pushing the cap or pulling the cap with the actuator causes the transition of the expandable basket portion between the low and expanded profile configurations.

16. The medical system of claim 1 wherein the stylet includes a cannulated shaft and a plurality of curved tines configured to contact the inner wall of the tool, wherein the plurality of curved tines are movable relative to the cannulated shaft.

17. The medical system of claim 16 wherein the cannulated shaft is movable distally relative to the tines.

18. The medical system of claim 16 wherein the tines are extendable distally of the cannulated shaft or are extendable through openings along a longitudinal length of the cannulated shaft.

19-20. (canceled)

21. The medical system of claim 1 wherein the stylet includes a cannulated shaft and straight tines that are movable relative to a cannulated shaft to flare out distally from the cannulated shaft.

22. The medical system of claim 21 wherein the cannulated shaft is movable distally relative to the tines.

23. The medical system of claim 21 wherein the tines are advanceable distally of the cannulated shaft or are extendable through the cannulated shaft.

24-36. (canceled)
